(19) United States
(12) Patent Application Publication
(10) Pub. No.: US 2025/0354899 A1
(43) Pub. Date: Nov. 20, 2025

(54) APPARATUS AND METHOD FOR VACUUM WATER EXTRACTION FROM SOIL

(71) Applicant: NORTHWEST INSTITUTE OF ECO-ENVIRONMENT AND RESOURCES, CAS, Lanzhou (CN)

(72) Inventors: Caixia ZHANG, Lanzhou (CN); Zongsheng LI, Lanzhou (CN); Ningjing TAN, Lanzhou (CN)

(21) Appl. No.: 19/283,141

(22) Filed: Jul. 28, 2025

(30) Foreign Application Priority Data
Feb. 28, 2025 (CN) 202510229182.8

(51) Int. Cl. G01N 1/14; G01N 33/24

(52) U.S. Cl. CPC G01N 1/14; G01N 33/246; G01N 2001/1418

(57) ABSTRACT

A vacuum soil water extraction apparatus, including a bearing mechanism, a cooling mechanism and a temperature control mechanism. The bearing mechanism includes a carrier, a purging assembly, a vacuum assembly and a connecting pipe. The purging assembly, the vacuum assembly and the connecting pipe are provided on the carrier. The purging assembly and the vacuum assembly are both connected to the connecting pipe. The purging assembly is configured to blow air into the connecting pipe. The vacuum assembly is configured to vacuumize the connecting pipe. The sample tube and the water collection vessel are provided at two ends of the connecting pipe, respectively. The carrier is movably engaged with the cooling mechanism and the temperature control mechanism, such that the carrier is switchable between a first position and a second position. A vacuum soil water extraction method using such apparatus is also provided.

### CROSS-REFERENCE TO RELATED APPLICATIONS

This application claims the benefit of priority from Chinese Patent Application No. 202510229182.8, filed on Feb. 28, 2025. The content of the aforementioned application, including any intervening amendments made thereto, is incorporated herein by reference in its entirety.

### TECHNICAL FIELD

This application relates to environmental monitoring technologies, and more particularly to an apparatus and method for vacuum water extraction from soil.

### BACKGROUND

Soil water extraction is a technique for separating and collecting water from soil samples, and is critical for researches in the fields of agriculture, environmental science, geological science, and engineering. This technique enables the measurement of soil water content, and is thus of great significance for the evaluation of soil properties, design of an irrigation plan, and monitoring of dynamic soil moisture changes. Moreover, it also supports soil-water retention curve analysis, soil structure/quality assessment, environmental contamination research, agricultural management and engineering applications. In the prior art, the water extraction from a collected soil sample is generally performed manually.

The existing soil water extraction methods at least suffer from labor-intensive and cumbersome manual operation, low efficiency, and excessive resource consumption such as liquid nitrogen.

### SUMMARY

An object of the disclosure is to provide an apparatus and method for vacuum water extraction from soil, which can improve the degree of automation, reduce labor intensity, increase operational efficiency, and enhancing the reliability and accuracy of experimental data.

Technical solutions of the present disclosure are described as follows.

In a first aspect, this application provides an apparatus for vacuum water extraction from soil, the apparatus being adapted to be compatible with a sample tube and a water collection vessel, and the apparatus comprising:
a bearing mechanism;

a cooling mechanism; and
a temperature control mechanism;
wherein the bearing mechanism comprises a carrier, a purging assembly, a vacuum assembly and a connecting pipe;
the purging assembly, the vacuum assembly and the connecting pipe are provided on the carrier;
the purging assembly and the vacuum assembly are connected to the connecting pipe;
the purging assembly is configured to blow air into the connecting pipe;
the vacuum assembly is configured to vacuumize the connecting pipe;
a first end of the connecting pipe is configured to be connected to the sample tube, and a second end of the connecting pipe is configured to be connected to the water collection vessel;
the carrier is movably engaged with the cooling mechanism and the temperature control mechanism, such that the carrier is switchable between a first position and a second position;
in response to a case that the carrier is in the first position, the cooling mechanism is configured to cool the sample tube; and
in response to a case that the carrier is in the second position, the temperature control mechanism is configured to simultaneously heat the sample tube and cool the water collection vessel.

In some embodiments, the carrier comprises a first driver, a second driver, and a positioning plate assembly; the first driver is connected to the second driver; the second driver is connected to the positioning plate assembly; and the connecting pipe is provided on the positioning plate assembly;
the first driver is configured to drive the second driver and the positioning plate assembly to rotate synchronously with respect to the cooling mechanism and the temperature control mechanism; and
the second driver is configured to drive the positioning plate assembly to move up and down with respect to the cooling mechanism and the temperature control mechanism.

In some embodiments, the positioning plate assembly comprises an upper pressing plate and a lower pressing plate;
the upper pressing plate is connected to the lower pressing plate;
the upper pressing plate and the lower pressing plate are configured to cooperate with each other to clamp the connecting pipe;
the first end and the second of the connecting pipe are configured to extend out of an area between the upper pressing plate and the lower pressing plate;
the upper pressing plate or the lower pressing plate is connected to the second driver; and
the second driver is configured to drive the upper pressing plate and the lower pressing plate to synchronously move up and down.

In some embodiments, the connecting pipe is configured as a U-shaped pipe;
the connecting pipe comprises a first pipe section, a second pipe section and a third pipe section connected in sequence;
the first pipe section and the third pipe section are configured to extend in the same direction;
the upper pressing plate and the lower pressing plate are configured to cooperate with each other to clamp the second pipe section;
the upper pressing plate is provided above the lower pressing plate; and
the first pipe section and the third pipe section are located on a side of the upper pressing plate adjacent to the lower pressing plate.

In some embodiments, the lower pressing plate is provided with an avoidance hole; the first pipe section is configured to pass through the avoidance hole; the third pipe section is configured to extend out of an edge of the lower pressing plate; the first pipe section is connected to the water collection vessel; and the third pipe section is connected to the sample tube.

In some embodiments, the temperature control mechanism comprises a heating tank and a cooling tank separately arranged; the heating tank is provided with a heating cotton configured to contact and heat the sample tube; and the cooling tank is configured to contain liquid nitrogen for cooling the water collection vessel.

In some embodiments, the sample tube is configured as a bent tube; and the sample tube is located on an outer side of the water collection vessel;

an end of the sample tube away from the connecting pipe is configured to face outward; and a bottom of the sample tube is lower than a bottom of the water collection vessel.

In some embodiments, a blocking cotton is provided within the sample tube; and the blocking cotton is configured to allow air to pass through and prevent a soil sample within the sample tube from exiting the sample tube during a vacuumization process.

In a second aspect, this application provides a vacuum soil water extraction method using the apparatus described above, comprising:

(S100) introducing air into the connecting pipe through the purging assembly;

(S200) mounting the sample tube at the first end of the connecting pipe, and mounting the water collection vessel at the second end of the connecting pipe; adding a soil sample into the end of the sample tube away from the connecting pipe followed by sealing; and vacuumizing the connecting pipe, the sample tube, and the water collection vessel using the vacuum assembly;

(S300) when the carrier is in the first position, cooling the sample tube using the cooling mechanism; and

(S400) when the carrier is in the second position, heating the sample tube and cooling the water collection vessel simultaneously using the temperature control mechanism.

Compared to the prior art, the present disclosure has the following beneficial effects.

The apparatus provided herein integrates the purging assembly, the vacuum assembly and the connecting pipe on the carrier, resulting in a compact structure and small overall size, which facilitates centralized management. The connecting pipe is positioned by the carrier, ensuring stable and reliable alignment. When the sample tube is mounted at the first end of the connecting pipe, and the water collection vessel is mounted at the second end of the connecting pipe, slippage is unlikely to occur, making installation faster and more labor-efficient. Purging and vacuumizing operations can be performed without additional positioning of the connecting pipe, which improves operational flexibility and convenience. Moreover, since the sample tube and the water collection vessel are both positioned by the connecting pipe, their relative alignment is more accurate, which benefits the subsequent water extraction process. By adjusting the position of the carrier, the relative position of the sample tube and the water collection vessel with respect to the cooling mechanism and the temperature control mechanism can be precisely controlled. This allows convenient and accurate cooling or heating of the sample tube, as well as effective cooling of the water collection vessel. In addition, automated operation minimizes manual intervention, reducing variability between the cooling and heating of the sample tube and the cooling of the water collection vessel. As a result, operational errors are reduced, and the accuracy of the experimental results is significantly improved.

### BRIEF DESCRIPTION OF THE DRAWINGS

In order to illustrate the technical solutions in the present disclosure more clearly, the accompanying drawings needed in the description of the embodiments will be briefly described below. It is evident that presented in the following accompanying drawings are only some embodiments of the present disclosure, instead of all embodiments. For those of ordinary skill in the art, other accompanying drawings can be obtained based on these accompanying drawings without making creative effort.

FIG. 1 schematically shows an apparatus for vacuum water extraction from soil according to an embodiment of the present disclosure;

FIG. 2 is a partial schematic view of the apparatus according to an embodiment of the present disclosure;

FIG. 3 schematically shows a pipeline assembly according to an embodiment of the present disclosure;

FIG. 4 schematically shows a cooperation between a connecting pipe, a water collection vessel and a sample tube according to an embodiment of the present disclosure;

FIG. 5 schematically shows the sample tube according to an embodiment of the present disclosure; and

FIG. 6 schematically shows a cooling mechanism and a temperature control mechanism according to an embodiment of the present disclosure.

In the figures: 001—sample tube; 011—vertical tube segment; 012—horizontal tube segment; 013—rubber stopper; 014—blocking cotton; 015—conduit; 016—pull wire; 017—metal mesh basket; 002—water collection vessel; 100—bearing mechanism; 110—carrier; 111—first driver; 112—second driver; 113—positioning plate assembly; 1131—upper pressing plate; 1132—lower pressing plate; 1133—avoidance hole; 1134—sliding sleeve; 120—purging assembly; 121—protective box; 122—nitrogen cylinder; 123—first gas pipe; 124—first valve; 125—slide rail; 130—vacuum assembly; 131—vacuum pump; 132—second gas pipe; 133—second valve; 140—pipeline assembly; 141—main pipe; 142—docking pipe; 143—connecting pipe; 1431—first pipe section; 1432—second pipe section; 1433—third pipe section; 200—cooling mechanism; 300—temperature control mechanism; 310—heating tank; 320—cooling tank; 330—heating cotton; and 400—base.

### DETAILED DESCRIPTION OF EMBODIMENTS

In order to make the objects, technical solutions and advantages of the present disclosure clearer, the present disclosure will be described clearly and completely below in conjunction with the accompanying drawings and embodiments. Obviously, described herein are merely some embodiments of the present disclosure, rather than all embodiments. The components of embodiments of the present disclosure described and shown in the accompanying drawings may be arranged and designed in a variety of different configurations.

Thus, the following detailed description is merely illustrative of selected embodiments of the present disclosure in the accompanying drawings, is not intended to limit the scope of the present disclosure. Based on the embodiments in the present disclosure, all other embodiments obtained by those of ordinary skill in the art without making creative effort shall fall within the scope of the present disclosure defined by the appended claims.

It should be noted that similar reference numerals and letters in the following accompanying drawings indicate similar items. Therefore, once an item is defined in one accompanying drawing, it does not require further definition or explanation in the subsequent accompanying drawings.

Additionally, in the description of the present disclosure, it should be noted that, as used herein, terms “up”, “down”, “inner” and “outer” are based on the those shown in the accompanying drawings. These terms are solely for the convenience of describing the present disclosure, and are not intended to indicate or imply that the devices or components must have specific orientations or be constructed and operated in specific orientations. Therefore, these terms should not be understood as limitations of the present disclosure.

Furthermore, as used herein, terms “first” and “second” are merely used to distinguish technical features, rather than indicating or implying their relative importance.

It should be noted that, where there is no conflict, the features of the embodiments of the present disclosure may be combined with one another.

In the prior art, the process of soil water extraction is performed manually throughout. The operation involves numerous steps, resulting in high labor intensity and low efficiency. Moreover, the methods used by different operators, or even by the same operator at different times, tend to vary, which leads to significant errors. The overall extraction process is time-consuming, labor-intensive and consumes considerable resources.

In view of this, the present disclosure provides an apparatus for vacuum water extraction from soil, which can improve the degree of automation, reduce labor intensity, increase operational efficiency, minimize errors, and enhance the reliability and accuracy of experimental results.

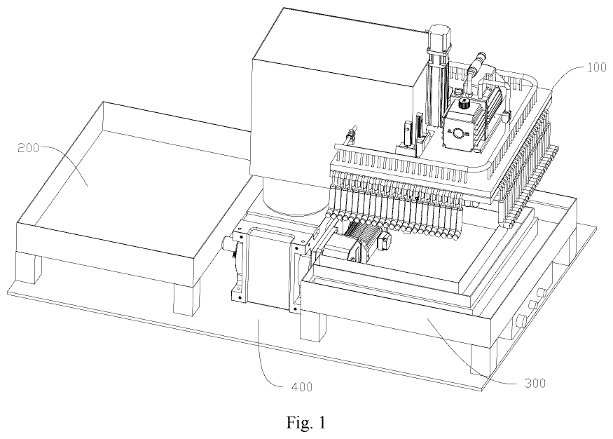

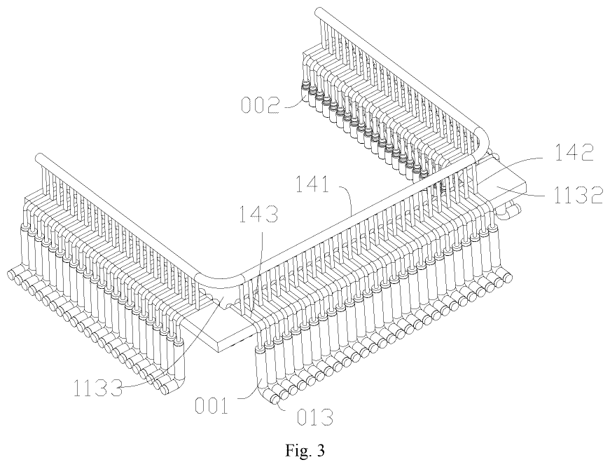

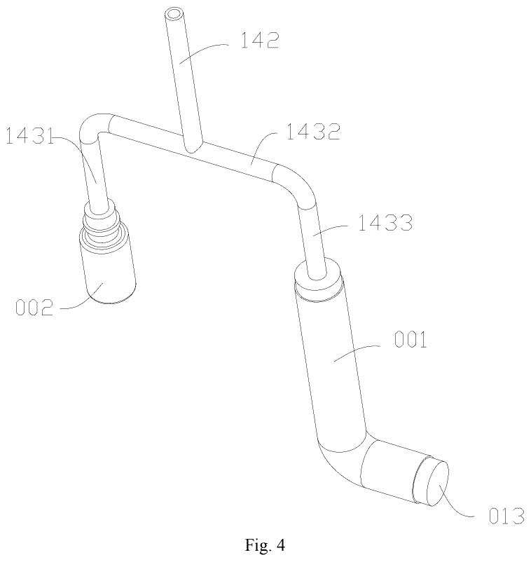

As shown in FIGS. 1-6, an embodiment of the present disclosure provides an apparatus for vacuum water extraction from soil, which is adapted to be compatible with a sample tube 001 and a water collection vessel 002. The apparatus includes a bearing mechanism 100, a cooling mechanism 200 and a temperature control mechanism 300. The bearing mechanism 100 includes a carrier 110, a purging assembly 120, a vacuum assembly 130 and a connecting pipe 143. The purging assembly 120, the vacuum assembly 130 and the connecting pipe 143 are provided on the carrier 110. The purging assembly 120 and the vacuum assembly 130 are connected to the connecting pipe 143. The purging assembly 120 is configured to blow air into the connecting pipe 143. The vacuum assembly 130 is configured to vacuumize the connecting pipe 143. A first end of the connecting pipe 143 is configured to be connected to the sample tube 001, and a second end of the connecting pipe 143 is configured to be connected to the water collection vessel 002. The carrier 110 is movably engaged with the cooling mechanism 200 and the temperature control mechanism 300, such that the carrier 110 is switchable between a first position and a second position. When the carrier 110 is in the first position, the cooling mechanism 200 is configured to cool the sample tube 001. When the carrier 110 is in the second position, the temperature control mechanism 300 is configured to simultaneously heat the sample tube 001 and cool the water collection vessel 002.

As described above, the apparatus provided herein operates as follows.

First, the purging assembly 120 is activated while the vacuum assembly 130 remains closed. The purging assembly 120 blows air into the connecting pipe 143 to remove residual air and impurities. The purging assembly 120 blows an inert gas (e.g. nitrogen gas) into the connecting pipe 143. Next, the purging assembly 120 is deactivated, the sample tube 001 is mounted at the first end of the connecting pipe 143, and the water collection vessel 002 is mounted at the second end of the connecting pipe 143 in a sealed manner. A suitable amount of soil sample is added into the sample tube 001 from an end of the sample tube 001 away from the connecting pipe 143. The end of the sample tube 001 away from the connecting pipe 143 is then sealed using a sealing plug. Afterward, the vacuum assembly 130 is activated to vacuumize the connecting pipe 143, the sample tube 001, and the water collection vessel 002, thereby establishing a vacuum environment. Vacuumizing helps to remove water and impurities from the residual air inside the connecting pipe 143, the sample tube 001, and the water collection vessel 002, ensuring the purity of the extracted sample. The carrier 110 is positioned in the first position, where the cooling mechanism 200 cools the sample tube 001 and the soil sample contained therein. Liquid nitrogen is used to cool the soil sample, which facilitates the separation of water from the soil sample and improves the extraction efficiency. Subsequently, the carrier 110 is switched from the first position to the second position. In the second position, the temperature control mechanism 300 heats the sample tube 001 and cool the water collection vessel 002 simultaneously. The sample tube 001 is heated using a heating element while the water collection vessel 002 is cooled using liquid nitrogen, thereby increasing the temperature difference to enhance the efficiency of soil water extraction.

It should be understood that the purging assembly 120, the vacuum assembly 130 and the connecting pipe 143 are provided on the carrier 110, resulting in a compact structure with a small footprint, which facilitates centralized management. The connecting pipe 143 is positioned and supported by the carrier 110, thereby ensuring stable and reliable alignment. When the sample tube 001 is mounted at the first end of the connecting pipe 143, and the water collection vessel 002 is mounted at the second end of the connecting pipe 143, slippage is effectively prevented, and the installation process becomes faster, easier, and more efficient. The entire operation process is highly automated, requires minimal human intervention, involves low labor intensity, and exhibits reduced operational error. As a result, the accuracy and reliability of experimental outcomes can be significantly improved.

The following embodiments are provided to illustrate details of the apparatus for vacuum water extraction from soil described herein.

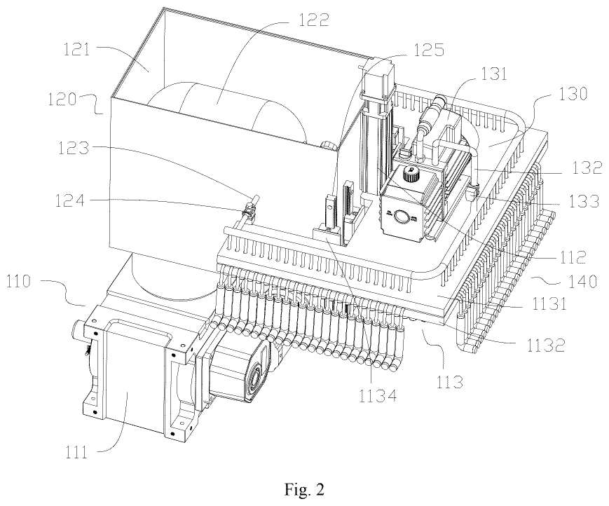

As shown in FIGS. 1-2, in this embodiment, the apparatus includes the bearing mechanism 100, the cooling mechanism 200, the temperature control mechanism 300, and a base 400. The bearing mechanism 100, the cooling mechanism 200, and the temperature control mechanism 300 are provided on the base 400, resulting in a compact overall structure with a small footprint that facilitates maintenance. Meanwhile, the bearing mechanism 100 is configured to cooperate with the cooling mechanism 200 and the temperature control mechanism 300 to perform water extraction operations on the soil sample.

The bearing mechanism 100 includes the carrier 110, the purging assembly 120, the vacuum assembly 130, and a pipeline assembly 140. The purging assembly 120, the vacuum assembly 130 and the pipeline assembly 140 are all operably associated with the carrier 110. The purging assembly 120 and the vacuum assembly 130 are each connected to the pipeline assembly 140.

Referring to FIG. 2, the carrier 110 includes a first driver 111, a second driver 112 and a positioning plate assembly 113. The first driver 111 is connected to the second driver 112, and the second driver 112 is connected to the positioning plate assembly 113. The first driver 111 is configured to drive the second driver 112 and the positioning plate assembly 113 to rotate synchronously with respect to the cooling mechanism 200 and the temperature control mechanism 300. The second driver 112 is configured to drive the positioning plate assembly 113 to move up and down with respect to the cooling mechanism 200 and the temperature control mechanism 300. In some embodiments, the first driver 111 is an electric motor or a motor. The first driver 111 is fixedly provided on the base 400. A rotating shaft of the first driver 111 is configured to extend vertically, so as to drive the second driver 112 and the positioning plate assembly 113 to rotate within a horizontal plane, thereby adjusting the position of the positioning plate assembly 113 in the horizontal plane. In some embodiments, the second driver 112 is a stepper motor, and is configured to drive the positioning plate assembly 113 to reciprocate in the vertical direction. The stepper motor also facilitates precise and flexible control.

Furthermore, the positioning plate assembly 113 includes an upper pressing plate 1131 and a lower pressing plate 1132. The upper pressing plate 1131 and the lower pressing plate 1132 are arranged in a stacked manner. The upper pressing plate 1131 is fixedly connected to the lower pressing plate 1132 by bolts. A clamping space is formed between the upper pressing plate 1131 and the lower pressing plate 1132. The upper pressing plate 1131 or the lower pressing plate 1132 is connected to a telescopic end of the second driver 112. During normal operation, the upper pressing plate 1131 is located above the lower pressing plate 1132, or, the upper pressing plate 1131 is located on a side of the lower pressing plate 1132 away from the base 400. In an embodiment, the lower pressing plate 1132 includes a first side, a second side, a third side and a fourth side sequentially connected end to end. The first side is provided with a sliding sleeve 1134. In some embodiments, the sliding sleeve 1134 is provided in plurality to enhance the stability of the positioning plate assembly 113 during sliding movement. The lower pressing plate 1132 is provided with an avoidance hole 1133 extending through a surface of the lower pressing plate 1132.

As shown in FIG. 2, the purging assembly 120 includes a protective box 121, a nitrogen cylinder 122, a first gas pipe 123 and a first valve 124. The protective box 121 is fixedly provided to the rotating shaft of the first driver 111. The nitrogen cylinder 122 is provided within the protective box 121. A gas outlet of the nitrogen cylinder 122 is communicated with the first gas pipe 123. The first gas pipe 123 is provided with the first valve 124. The first valve 124 is configured to control the on/off state of the first gas pipe 123, so as to control a gas flow rate in the first gas pipe 123. An outer surface of the protective box 121 is provided with a sliding rail 125. The number of sliding rails 125 corresponds to the number of sliding sleeves 1134 in a one-to-one correspondence. Each sliding rail 125 is slidably engaged with a corresponding sliding sleeve 1134. The cooperation between the sliding rails 125 and the sliding sleeves 1134 improves the stability of the positioning plate assembly 113. To avoid interference between the positioning plate assembly 113 and the first gas pipe 123 during vertical movement, the first gas pipe 123 is configured as a flexible hose to allow for adaptive deformation.

In some embodiments, the vacuum assembly 130 includes a vacuum pump 131, a second gas pipe 132 and a second valve 133. The vacuum pump 131 is provided on the upper pressing plate 1131. A gas outlet of the vacuum pump 131 is communicated with the second gas pipe 132. The second valve 133 is mounted to the second gas pipe 132, and is configured to control the on/off state of the second gas pipe 132, thereby controlling the vacuumizing efficiency.

Referring to FIGS. 2-4, the pipeline assembly 140 includes a main pipe 141 and three operating units. Both the first gas pipe 123 and the second gas pipe 132 are communicated with the main pipe 141. The main pipe 141 is configured as a U-shaped pipe. The main pipe 141 includes three pipe sections connected in sequence. The three pipe sections respectively correspond to the second side, the third side, and the fourth side of the lower pressing plate 1132. That is, a first pipe section of the three pipe sections of the main pipe 141 is configured to extend along the second side, a second pipe section of the three pipe sections of the main pipe 141 is configured to extend along the third side, and a third pipe section of the three pipe sections of the main pipe 141 is configured to extend along the fourth side. The three operating units respectively correspond to the three pipe sections. Each operating unit includes a plurality of docking pipes 142 and a plurality of connecting pipes 143. The docking pipes 142 and the connecting pipes 143 are provided in equal number and in one-to-one correspondence. The plurality of docking pipes 142 are evenly spaced apart along a length direction of the corresponding pipe section. A first end of each docking pipe 142 is communicated with the main pipe 141, and a second end of each docking pipe 142 is communicated with the corresponding connecting pipe 143. A connection between each docking pipe 142 and the corresponding connecting pipe 143 is located between the two ends of the connecting pipe 143, such that it does not interfere with the assembly of the sample tube 001 at the first end of the connecting pipe 143, and the water collection vessel 002 at the second end of the connecting pipe 143. During assembly, the main pipe 141 is provided above the upper pressing plate 1131. Each docking pipe 142 is configured to extend through the upper pressing plate 1131. The connecting pipes 143 are configured to be clamped between the upper pressing plate 1131 and the lower pressing plate 1132, and are configured to extend outward through the corresponding avoidance holes 1133.

In some embodiments, the connecting pipe 143 is configured as a bent pipe and is generally U-shaped. The connecting pipe 143 includes a first pipe section 1431, a second pipe section 1432 and a third pipe section 1433 sequentially connected. The first pipe section 1431 and the third pipe section 1433 are configured to extend in the same direction. The upper pressing plate 1131 and the lower pressing plate 1132 are configured to cooperate with each other to clamp the second pipe section 1432. The first pipe section 1431 and the third pipe section 1433 are located on a side of the upper pressing plate 1131 adjacent to the lower pressing plate 1132. The first pipe section 1431 is configured to extend through a corresponding avoidance hole 1133. The third pipe section 1433 is configured to extend out of an edge of a side of the lower pressing plate 1132, such that the third pipe section 1433 is provided at an outer side of the first pipe section 1431. The first pipe section 1431 is configured for mounting the water collection vessel 002, and the third pipe section 1433 is configured for mounting the sample tube 001. The sample tube 001 is provided on an outer side of the water collection vessel 002 to facilitate the loading and unloading of soil sample.

During the testing process, when purging is required, the first valve 124 is opened and the second valve 133 is closed. Nitrogen gas from the nitrogen cylinder 122 is discharged through the first gas pipe 123, enters the main pipe 141, and then flows through the docking pipes 142 into the connecting pipes 143, thereby purging and clearing debris from inside the main pipe 141, the connecting pipes 143, and the docking pipes 142. When vacuuming is required, the first valve 124 is closed and the second valve 133 is opened. The vacuum pump 131 is activated to evacuate the main pipe 141, the docking pipes 142, the connecting pipes 143, the water collection vessel 002 and the sample tube 001.

In addition, when the second driver 112 drives the positioning plate assembly 113 to move up and down, the positioning plate assembly 113 slides with respect to the guide rail 125 via the sliding sleeve 1134. The guide rail 125 is fixedly mounted to the protective box 121, which remains stationary during vertical movement. The first gas pipe 123 of the protective box 121 is configured to extend through the protective box 121 to be connected to the main pipe 141. As the positioning plate assembly 113 moves, it drives the main pipe 141, the docking pipes 142 and the connecting pipes 143 to move vertically together. As the first gas pipe 123 is a flexible hose, it deforms adaptively without causing interference or being pulled or damaged, ensuring safe and reliable operation.

In this embodiment, the cooling mechanism 200 includes a liquid nitrogen tank configured to hold a predetermined amount of liquid nitrogen. The carrier 110 lowers the sample tube 001, such that the sample tube 001 is inserted into the liquid nitrogen tank, thereby allowing the liquid nitrogen to cool the sample tube 001.

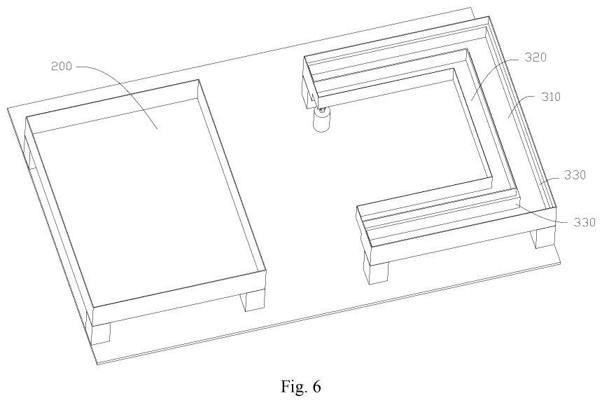

Referring to FIG. 6, in this embodiment, the temperature control mechanism 300 includes a heating tank 310 and a cooling tank 320 separately arranged. The heating tank 310 is provided with a heating cotton 330 configured to contact and heat the sample tube 001. The cooling tank 320 is configured to contain liquid nitrogen for cooling the water collection vessel 002. In some embodiments, the heating cotton 330 is configured as a plurality of positioning recesses. The number of positioning recesses corresponds to the number of sample tubes 001, ensuring that each sample tube 001 is inserted into a corresponding one of the plurality of positioning recesses for effective heating.

It should be understood that the heating cotton 330 can be formed by embedding fine electric heating wires or conductive materials such as carbon fibers into a fabric base. When energized, the heating cotton 330 converts electrical energy into thermal energy to perform heating.

To prevent mutual thermal interference, a heat insulation wall is provided between the heating tank 310 and the cooling tank 320.

During the water extraction from the soil sample in the sample tube 001, the sample tube 001 is heated by the heating tank 310 while the water collection vessel002 is cooled by the cooling tank 320, thereby creating a temperature difference between the sample tube 001 and the water collection vessel 002. The water in the soil sample within the sample tube 001 evaporates upon heating to form water vapor, which flows through the third pipe section 1433 and the second pipe section 1432, and then enters the first pipe section 1431. The water collection vessel 002 is connected to the first pipe section 1431. Due to the lower temperature of the water collection vessel 002, the water vapor condenses upon entering and is collected as liquid water within the water collection vessel 002.

To ensure that the water vapor smoothly reaches the water collection vessel 002, a valve can be provided at a connection between the docking pipe 142 and the second pipe section 1432. During water extraction, the valve is closed to prevent the water vapor from entering the docking pipe 142. The valve can be an electromagnetic valve to facilitate precise control.

In addition to the simple and reasonable structure, the apparatus provided herein also has high degree of integration, which can facilitate the subsequent maintenance and repair. Moreover, the apparatus also possesses enhanced automation level, which contributes to the improved accuracy of test results.

Referring to FIGS. 1-6, the first end of the connecting pipe 143 is connected to the sample tube 001, and the second end of the connecting pipe 143 is connected to the water collection vessel 002. A bottom of the sample tube 001 is lower than a bottom of the water collection vessel 002. By configuring the bottom of the sample tube 001 to be lower than the bottom of the water collection vessel 002, when the second driver 112 drives the carrier 110 and the connecting pipe 143 to descend, the sample tube 001 and the water collection vessel 002 descend together. The sample tube 001 comes into contact with the liquid nitrogen in the liquid nitrogen tank, while the water collection vessel 002 remains out of contact with the liquid nitrogen. This configuration reduces the consumption of liquid nitrogen and lowers operating costs. At the same time, since the water collection vessel 002 does not contact the liquid nitrogen, it does not interfere with the cooling of the soil sample in the sample tube 001, thereby improving the efficiency and effectiveness of the cooling process.

In some embodiments, the sample tube 001 is configured as a bent tube, and is provided on the outer side of the water collection vessel 002. The end of the sample tube 001 away from the connecting pipe 143 is configured to face outward. In some embodiments, the sample tube 001 is configured as an L-shaped tube. The sample tube 001 includes a vertical tube segment 011 and a horizontal tube segment 012 that are connected to each other. The vertical tube segment 011 is communicated with the connecting pipe 143. An end opening of the horizontal tube segment 012 is configured to face outward. The end opening of the horizontal tube segment 012 is configured to receive the soil sample. After the soil sample is added, the end opening of the horizontal tube segment 012 is sealed with a rubber stopper 013.

A blocking cotton 014 is provided within the sample tube 001. The blocking cotton 014 is configured to allow air to pass through and prevent the soil sample within the sample tube 001 from exiting the sample tube 001 during a vacuumization process. The blocking cotton 014 is provided in the vertical tube segment 011 of the sample tube 001.

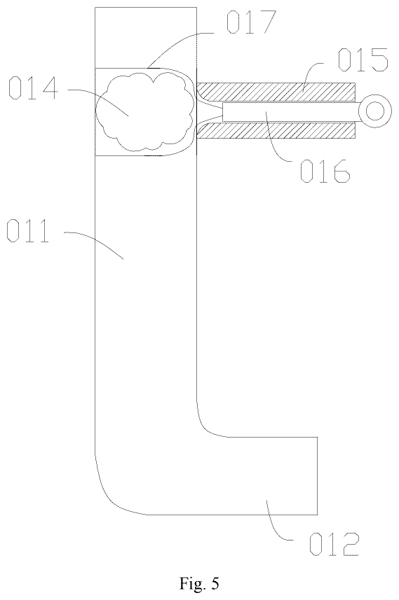

Referring to FIG. 5, in order to prevent the blocking cotton 014 from absorbing a portion of the evaporated water and thereby affecting the accuracy of water extraction, a wall of the vertical tube segment 011 is provided with a conduit 015. A pull wire 016 is slidably provided within the conduit 015, and is in dynamic sealing contact with the conduit 015. An end of the pull wire 016 is provided with a metal mesh basket 017. The metal mesh basket 017 is configured to contract, and is capable of expanding when located within the vertical tube segment 011, so as to accommodate the blocking cotton 014 in a fluffed state. The pull wire 016 is configured to drive the metal mesh basket 017, together with the blocking cotton 014, from within the vertical tube segment 011 into the conduit 015, such that the metal mesh basket 017 is contracted and cooperates with the blocking cotton 014 to sealedly block the conduit 015. At this time, the blocking cotton 014 is compressed, significantly reducing its porosity and thereby effectively minimizing the absorption of water vapor.

The apparatus disclosed herein for vacuum water extraction from soil has simple and flexible operation, and enhances the accuracy and reliability of test results.

The present disclosure further provides a vacuum soil water extraction method using the apparatus described herein. The method includes the following steps.
(S100) Air is introduced into the connecting pipe 143 through the purging assembly 120 to remove impurities inside the connecting pipe 143.
(S200) The sample tube 001 is mounted at the first end of the connecting pipe, and the water collection vessel 002 is mounted at the second end of the connecting pipe 143. A soil sample is added into the end of the sample tube 001 away from the connecting pipe 143, and the end of the sample tube 001 away from the connecting pipe 143 is sealed. The connecting pipe 143, the sample tube 001 and the water collection vessel 002 are vacuumized by the vacuum assembly 130.
(S300) When the carrier 110 is in the first position, the sample tube 001 is cooled by the cooling mechanism 200. Liquid nitrogen is used to cool the sample tube 001. As a result, water contained in the soil sample within the sample tube 001 is more readily separated, thereby improving the extraction efficiency.
(S400) When the carrier 110 is in the second position, the sample tube 001 is heated by the temperature control mechanism 300 while the water collection vessel 002 is simultaneously cooled. In this process, water within the soil sample is driven out and collected. The purging assembly 120 and the vacuum assembly 130 remain deactivated during this step.

The method provided herein is characterized by flexible operation, a high degree of automation, high efficiency, minimal manual involvement, low labor intensity, and improved accuracy and the reliability of experimental results.

Described embodiments are merely illustrative, and are not intended to limit the scope of the present disclosure. It should be understood that various modifications, changes and replacements made by those skilled in the art without departing from the spirit of the disclosure shall fall within the scope of the present disclosure defined by the appended claims.

What is claimed is:

1. An apparatus for vacuum water extraction from soil, the apparatus being adapted to be compatible with a sample tube and a water collection vessel, and the apparatus comprising:
a bearing mechanism;
a cooling mechanism; and
a temperature control mechanism;
wherein the bearing mechanism comprises a carrier, a purging assembly, a vacuum assembly and a connecting pipe;
the purging assembly, the vacuum assembly and the connecting pipe are provided on the carrier;
the purging assembly and the vacuum assembly are connected to the connecting pipe;
the purging assembly is configured to blow air into the connecting pipe;
the vacuum assembly is configured to vacuumize the connecting pipe;
a first end of the connecting pipe is configured to be connected to the sample tube, and a second end of the connecting pipe is configured to be connected to the water collection vessel;
the carrier is movably engaged with the cooling mechanism and the temperature control mechanism, such that the carrier is switchable between a first position and a second position;
in response to a case that the carrier is in the first position, the cooling mechanism is configured to cool the sample tube;
in response to a case that the carrier is in the second position, the temperature control mechanism is configured to simultaneously heat the sample tube and cool the water collection vessel;
the sample tube is configured as a bent tube; and the sample tube is located on an outer side of the water collection vessel;
an end of the sample tube away from the connecting pipe is configured to face outward; and
a bottom of the sample tube is lower than a bottom of the water collection vessel.

2. The apparatus according to claim 1, wherein the carrier comprises a first driver, a second driver and a positioning plate assembly; the first driver is connected to the second driver; the second driver is connected to the positioning plate assembly; and the connecting pipe is provided on the positioning plate assembly;
the first driver is configured to drive the second driver and the positioning plate assembly to rotate synchronously with respect to the cooling mechanism and the temperature control mechanism; and
the second driver is configured to drive the positioning plate assembly to move up and down with respect to the cooling mechanism and the temperature control mechanism.

3. The apparatus according to claim 2, wherein the positioning plate assembly comprises an upper pressing plate and a lower pressing plate;
the upper pressing plate is connected to the lower pressing plate;
the upper pressing plate and the lower pressing plate are configured to cooperate with each other to clamp the connecting pipe;
the first end and the second end of the connecting pipe are configured to extend out of an area between the upper pressing plate and the lower pressing plate;
the upper pressing plate or the lower pressing plate is connected to the second driver; and
the second driver is configured to drive the upper pressing plate and the lower pressing plate to synchronously move up and down.

4. The apparatus according to claim 3, wherein the connecting pipe is configured as a U-shaped pipe;
the connecting pipe comprises a first pipe section, a second pipe section and a third pipe section connected in sequence;
the first pipe section and the third pipe section are configured to extend in the same direction;
the upper pressing plate and the lower pressing plate are configured to cooperate with each other to clamp the second pipe section;
the upper pressing plate is provided above the lower pressing plate; and
the first pipe section and the third pipe section are located on a side of the upper pressing plate adjacent to the lower pressing plate.

5. The apparatus according to claim 4, wherein the lower pressing plate is provided with an avoidance hole; the first pipe section is configured to pass through the avoidance hole; the third pipe section is configured to extend out of an edge of the lower pressing plate; the first pipe section is connected to the water collection vessel; and the third pipe section is connected to the sample tube.

6. The apparatus according to claim 1, wherein the temperature control mechanism comprises a heating tank and a cooling tank separately arranged; the heating tank is provided with a heating cotton configured to contact and heat the sample tube; and the cooling tank is configured to contain liquid nitrogen for cooling the water collection vessel.

7. The apparatus according to claim 1, wherein a blocking cotton is provided within the sample tube; and the blocking cotton is configured to allow air to pass through and prevent a soil sample within the sample tube from exiting the sample tube during a vacuumization process.

8. A vacuum soil water extraction method using the apparatus according to any one of claims 1-7, comprising:
(S100) introducing air into the connecting pipe through the purging assembly;
(S200) mounting the sample tube at the first end of the connecting pipe, and mounting the water collection vessel at the second end of the connecting pipe; adding a soil sample into the end of the sample tube away from the connecting pipe followed by sealing; and vacuumizing the connecting pipe, the sample tube and the water collection vessel using the vacuum assembly;
(S300) when the carrier is in the first position, cooling the sample tube using the cooling mechanism; and
(S400) when the carrier is in the second position, heating the sample tube and cooling the water collection vessel simultaneously using the temperature control mechanism.
